(19) United States
(12) Patent Application Publication
(10) Pub. No.: US 2025/0221681 A1
(43) Pub. Date: Jul. 10, 2025

(54) AUTOMATICALLY DETERMINING AN OPERATION MODE FOR AN X-RAY IMAGING SYSTEM

(71) Applicant: Siemens Healthineers AG, Forchheim (DE)

(72) Inventors: Arpitha Ravi, Erlangen (DE); Philipp Bernhardt, Forchheim (DE)

(21) Appl. No.: 19/015,619

(22) Filed: Jan. 9, 2025

(30) Foreign Application Priority Data
Jan. 10, 2024 (EP) 24151101

(51) Int. Cl. A61B 6/00; G06V 10/80

(52) U.S. Cl. CPC A61B 6/545; A61B 6/4441; G06V 10/806

(57) ABSTRACT

For automatically determining an operation mode for an X-ray imaging system that includes an X-ray source and an X-ray detector mounted on a C-arm, angulation data defining an angulation state of the C-arm is received, and at least one X-ray image depicting an object according to the angulation state is received. The operation mode for the X-ray imaging system is selected as one of two or more predefined operation modes by applying a trained machine learning model for classification to input data. The input data includes the at least one X-ray image and the angulation data.

This application claims the benefit of European Patent Application No. EP24151101, filed on Jan. 10, 2024, which is hereby incorporated by reference in its entirety.

### BACKGROUND

The present embodiments are directed to a computer implemented method for automatically determining an operation mode for an X-ray imaging system, a method for X-ray imaging, and a computer implemented training method for training a machine learning model for classification for use in the computer implemented method. The present embodiments are further directed to a corresponding data processing apparatus, an X-ray imaging system including the data processing apparatus, and to corresponding computer program products.

In X-ray medical imaging, it is important that the X-ray imaging system is configured properly for the requested task. For example, a static situation such as bone images of legs or arms allows for long exposure times, whereas imaging of the lung requires considering motion blurring. Also, within certain disciplines, such as cardiac imaging of the coronary vessels, selecting a certain parametrization of the X-ray imaging system may be beneficial, since each projection or task within a diagnostic or interventional procedure has specific requirements.

In order to assist a user in achieving the optimum system condition, users may have the option to choose a comprehensive package of parameter lists, also denoted as organ programs, OGPs, or operating modes, which are settings specifically tailored to accomplish a given task. However, for various reasons, users may select a generic OGP, which is applicable for various use cases and therefore necessarily represents a compromise between the requirements of different use cases. Consequently, the selected OGP may not be optimal for the actual use case, which leads to a reduced performance of the X-ray imaging system and/or a reduced quality of the resulting X-ray images.

ResNet is a popular architecture for artificial neural networks, ANN (e.g., convolutional neural networks, CNN), which was introduced in the publication K. He et al., “Deep Residual Learning for Image Recognition,” Proceedings of the IEEE Conference on Computer Vision and Pattern Recognition (CVPR), 2016, pp. 770-778. ResNet models are widely used for deep learning tasks due to their ability to enable the training of very deep neural networks. The ResNet-50 is a ResNet with a depth of 50 layers.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a possibility to automatically determine an operation mode for an X-ray imaging system is provided.

The present embodiments are based on the idea to treat the selection of an operation mode as a classification problem. A trained machine learning model, MLM, is used to carry out the classification depending on the angulation state of the C-arm and at least one X-ray image taken according to the angulation state.

According to an aspect of the present embodiments, a computer implemented method for automatically determining an operation mode for an X-ray imaging system is provided. The X-ray imaging system includes an X-ray source mounted on a C-arm of the X-ray imaging system and an X-ray detector mounted on the C-arm. Angulation data defining an angulation state of the C-arm is received. At least one X-ray image depicting an object according to the angulation state is received. The operation mode for the X-ray imaging system is selected as one of two or more predefined operation modes by applying a trained machine learning model, MLM, for classification to input data. The input data includes the at least one X-ray image and the angulation data.

Unless stated otherwise, all acts of the computer-implemented method may be performed by a data processing apparatus that includes at least one computing unit. For example, the at least one computing unit is configured or adapted to perform the acts of the computer-implemented method. For this purpose, the at least one computing unit may, for example, store a computer program including instructions that, when executed by the at least one computing unit, cause the at least one computing unit to execute the computer-implemented method.

In case the at least one computing unit includes two or more computing units, certain acts carried out by the at least one computing unit may be understood such that different computing units carry out different acts or different parts of an act. For example, it is not required that each computing unit carries out the acts completely. In other words, carrying out the acts may be distributed amongst the two or more computing units.

In general terms, a trained MLM may mimic cognitive functions that humans associate with other human minds. For example, by training based on training data, the MLM may be able to adapt to new circumstances and to detect and extrapolate patterns. Another term for a trained MLM is “trained function”.

In general, parameters of an MLM may be adapted or updated by training. For example, supervised training, semi-supervised training, unsupervised training, reinforcement learning, and/or active learning may be used. Further, representation learning, also denoted as feature learning, may be used. For example, the parameters of the MLMs may be adapted iteratively by a number of steps of training. For example, within the training, a certain loss function, also denoted as cost function, may be minimized. For example, within the training of an ANN, the backpropagation algorithm may be used.

For example, an MLM may include an ANN, a support vector machine, a decision tree, and/or a Bayesian network, and/or the machine learning model may be based on k-means clustering, Q-learning, genetic algorithms, and/or association rules. For example, an ANN may be or include a deep neural network, a convolutional neural network, or a convolutional deep neural network. Further, an ANN may be an adversarial network, a deep adversarial network, and/or a generative adversarial network.

An operation mode defines, for example, at least one predefined setting for the X-ray imaging system. The at least one setting may include one or more operating parameters for the X-ray imaging system (e.g., the X-ray source, filters in the path of radiation, the X-ray detector, and so forth). The at least one setting may also include one or more parameters for image processing based on the data generated by the X-ray detector. The at least one setting may also include a respective specification whether or not certain functions are to be activated during the data acquisition or for the image processing and/or whether or not certain functions are to be activated during the data acquisition or for the image processing. An operation mode may, for example, correspond to a predefined organ program, OGP.

The X-ray imaging system may be configured according to the selected operation mode, and one or more further X-ray images depicting the object may be generated by the x-ray imaging system configured accordingly. The at least one X-ray image may also be generated by the X-ray imaging system using, for example, a preliminary operation mode or preliminary settings, respectively.

For example, in the context of X-ray-based imaging of vessels (e.g., cardiac imaging or imaging of the brain), a contrast agent may be used for generating the one or more further X-ray images. In this case, the at least one X-ray image may, for example, be generated before the contrast agent is applied.

The angulation state corresponds, for example, to the position of the X-ray source and the X-ray detector relative to the object or, in other words, to the direction of a straight line connecting the X-ray source to the X-ray detector (e.g., its center) in three dimensions. Commonly, the angulation state of the C-arm is given by two rotation angles. One of them is denoted as orbital rotation angle or cranial/caudal angle, respectively. The other one is denoted as angular rotation angle or left anterior oblique/right anterior oblique angle, respectively.

The present embodiments are based on the finding that, while neither the at least one X-ray image (e.g., without applying a contrast agent), nor the angulation state alone provide sufficient information to reliably predict the intended imaging procedure for generating the further X-ray images. The combination of the at least one X-ray image and the angulation state, however, has been found to allow for a very reliable prediction. Therefore, according to the present embodiments, the MLM uses both for the classification task.

According to a number of implementations, an information message informing a user about the selected operation mode is generated.

The information message may be output visually by a display and/or as a speech output, for example. The user may then initiate the configuration of the X-ray imaging system according to the selected operation mode or manually configure the X-ray imaging system accordingly.

According to a number of implementations, the X-ray imaging system is automatically configured according to the selected operation mode.

For example, one or more control signals may be generated depending on the selected operation mode, and the X-ray imaging system may be automatically configured depending on the one or more control signals.

In such implementations, the user does not have to carry out or initiate the configuration manually, which leads to a further increased reliability that the optimal operation mode is used and saves time for the user.

According to a number of implementations, configuring the X-ray imaging system according to the selected operation mode includes setting a value of an X-ray exposure time assigned to the selected operation mode and/or a value of a frame rate assigned to the selected operation mode and/or at least one image processing parameter assigned to the selected operation mode and/or activating or deactivating a function of the X-ray imaging system assigned to the selected operation mode.

The function may, for example, be a function for averaging two or more of the further X-ray images for avoiding motion artifacts or another function for image processing, for example.

Consequently, it is provided that the further X-ray images are generated with optimal quality for the actual use case.

According to a number of implementations, the MLM includes one or more artificial neural networks, ANNs, or one or more ANN modules of an ANN. The ANN or the ANN modules may, for example, be a convolutional neural network, CNN, or CNN modules, respectively.

According to a number of implementations, image features are generated by applying a first feature extraction module, which is, for example, an ANN module, of the MLM to the at least one X-ray image. The operation mode is selected depending on the image features.

The first feature extraction module may be given or be based on a known ANN architecture for extracting features from images. For example, the first feature extraction module may include a CNN (e.g., a residual neural network, ResNet, such as a ResNet-50). However, other types of ANN families, such as VGG, Inception, and so forth may also be used.

According to a number of implementations, angulation features are generated by applying a second feature extraction module that is, for example, an ANN module of the MLM to the angulation data. The operation mode is selected depending on the angulation features.

The second feature extraction module may be given or be based on a known ANN architecture for extracting features (e.g., a multi-layer perceptron (MLP)). Since the angulation data is given by relatively few numerical values, the second feature extraction module is, for example, not configured as a CNN.

According to a number of implementations, the operation mode is selected depending on the image features and the angulation features.

Consequently, the MLM includes two distinct feature extraction modules for extracting the relevant features from the two components of the input data (e.g., the at least one X-ray image and the angulation data, respectively). This approach has been found to predict the correct class for the operation mode very reliably.

According to a number of implementations, fused features are generated by fusing the image features with the angulation features by the MLM. The operation mode is selected depending on the fused features.

The fusion of the features may, for example, be carried out by concatenating the image features and the angulation features. In this way, the MLM (e.g., a further ANN module of the MLM) may use all the extracted features in a comprehensive manner to carry out the classification task.

According to a number of implementations, the operation mode is selected by applying at least one fully connected neural network layer of the MLM to the fused features.

Consequently, only a relatively low amount of memory resources and computational resources are required for the final classification.

According to a number of implementations, the angulation data includes the angular rotation angle and the orbital rotation angle of the C-arm. For example, the angulation data consists of the angular rotation angle and the orbital rotation angle.

Therefore, the complexity of the angulation data is particularly low, and the angulation data is very easy to retrieve during the prediction phase of the MLM as well as during the training phase.

According to a number of implementations, the two or more predefined operation modes include a first operation mode for imaging right coronary arteries and/or a second operation mode for imaging left coronary arteries and/or a third operation mode for imaging a left ventricle.

These types of X-ray imaging are particularly sensitive to the settings defined by the operation modes and therefore benefit particularly significantly from the present embodiments.

For use cases or use situations that may arise in a computer implemented method or another method according to the present embodiments and are not explicitly described herein, it may be provided that, in accordance with the method, an error message and/or a prompt for user feedback is output, and/or a default setting and/or a predetermined initial state is set.

According to a further aspect of the present embodiments, a method for X-ray imaging using an X-ray imaging system that includes an X-ray source and an X-ray detector mounted on a C-arm is provided. Therein, angulation data defining an angulation state of the C-arm is determined. The X-ray imaging system is configured (e.g., manually or automatically) according to at least one preliminary setting. At least one X-ray image depicting an object according to the angulation state and according to the at least one preliminary setting is generated by using the X-ray source and the X-ray detector. A computer implemented method for automatically determining an operation mode for the X-ray imaging system according to the present embodiments is carried out, for example, by at least one computing unit of the X-ray imaging system. The X-ray imaging system is configured (e.g., manually or automatically) according to the selected operation mode. A further X-ray image (e.g., according to the angulation state) is generated according to the selected operation mode (e.g., after configuring the X-ray imaging system according to the selected operation mode) by using the X-ray source and the X-ray detector.

For example, a contrast agent may be applied to the object after the at least one X-ray image is generated and before the further X-ray image is generated or while the further X-ray image is generated. Therefore, the total amount of contrast agent applied is not increased for generating the at least one X-ray image. Nevertheless, the combination of the at least one X-ray image with the angulation data still allows for a reliable prediction of the optimal operation mode.

According to a further aspect of the present embodiments, a computer implemented training method for training an MLM for classification for use in a computer implemented method for automatically determining an operation mode for an X-ray imaging system according to the present embodiments is provided. Therein, angulation training data defining a training angulation state of the C-arm is received. At least one training image depicting an object according to the training angulation state is received. A ground truth annotation for the angulation training data and the at least one training image is received. A predicted operation mode for the X-ray imaging system is selected as one of two or more predefined operation modes by applying the MLM to input training data, which includes the at least one X-ray training image and the training angulation data. The MLM is updated depending on the predicted operation mode and the ground truth annotation (e.g., using supervised training).

The described steps are, for example, understood as a single training run. A plurality of such runs may be carried out consecutively until a predetermined termination or convergence criterion regarding the second loss function is reached. Each set of at least one training image may be denoted as a training sample. The number of training samples may lie in the order of 100 or several times 100 (e.g., 400 to 1000). The number of training epochs may, for example, lie in the order 100-1000. The total number of training runs is, for example, given by the product of the number of training samples and the number of training epochs.

A training image of the at least one training image may be an X-ray image or a digitally reconstructed radiograph, DRR, or another simulated X-ray image.

The ground truth annotation is the one of the two or more predefined operation modes, which the MLM may predict if the MLM is fully trained. The ground truth annotations may be manually generated by clinical experts such as image quality specialists, radiological technologists, and/or medical physicists with a number of (e.g., several) years of experience in the field.

Unless stated otherwise, all acts of the computer-implemented method may be performed by a further data processing apparatus that includes at least one further computing unit. For example, the at least one further computing unit is configured or adapted to perform the acts of the computer-implemented training method. For this purpose, the at least one further computing unit may, for example, store a further computer program including instructions that, when executed by the at least one further computing unit, cause the at least one further computing unit to execute the computer implemented training method.

In case the at least one further computing unit includes two or more computing units, certain acts carried out by the at least one further computing unit may be understood such that different further computing units carry out different acts or different parts of an act. For example, it is not required that each further computing unit carries out the acts completely. In other words, carrying out the acts may be distributed amongst the two or more further computing units.

According to a number of implementations of the computer implemented training method, training image features are generated by applying a pre-trained first feature extraction module of the MLM to the at least one X-ray training image. Training angulation features are generated by applying a pre-trained second feature extraction module of the MLM to the angulation training data. Training fused features are generated by fusing the training image features with the training angulation features. The predicted operation mode is selected by applying at least one fully connected neural network layer of the MLM to the fused features. Network parameters (e.g., weighting factors and bias factors) of the at least one fully connected neural network layer are updated depending on the predicted operation mode and the ground truth annotation. Applying the at least one fully connected neural network layer may, for example, also include applying a Softmax function as a last act.

For example, a loss function (e.g., a classification loss function) such as a cross-entropy loss function is evaluated depending on the output of the at least one fully connected neural network layer and the ground truth annotation. The network parameters of the at least one fully connected neural network layer are updated depending on the value of the loss function (e.g., by using the backpropagation algorithm).

The first feature extraction module as well as the second feature extraction module are pre-trained in such implementations. In other words, the first feature extraction module and the second feature extraction module are frozen for training the at least one fully connected neural network layer, and their respective network parameters are not updated depending on the value of the loss function. Thus, the first feature extraction module and/or the second feature extraction module may be used for other purposes apart from the computer implemented method for automatically determining an operation mode for an X-ray imaging system according to the present embodiments as well.

In alternative implementations, the first feature extraction module and the second feature extraction module are not pre-trained and are trained together with the at least one fully connected neural network layer based on the loss function as described.

According to a number of implementations of the computer implemented method for automatically determining an operation mode for an X-ray imaging system, the MLM is trained by using the computer implemented training method according to the present embodiments.

In other words, carrying out the computer implemented method includes carrying out the computer implemented training method in such implementations.

In alternative implementations, the acts for carrying out the computer implemented training method are not part of the computer implemented method according to the present embodiments.

According to a further aspect of the present embodiments, a data processing apparatus including at least one computing unit that is configured to carry out a computer implemented method according to the present embodiments is provided.

A computing unit may, for example, be understood as a data processing device that includes processing circuitry. The computing unit may therefore, for example, process data to perform computing operations. This may also include operations to perform indexed accesses to a data structure (e.g., a look-up table (LUT)).

For example, the computing unit may include one or more computers, one or more microcontrollers, and/or one or more integrated circuits (e.g., one or more application-specific integrated circuits, ASIC, one or more field-programmable gate arrays, FPGA, and/or one or more systems on a chip, SoC). The computing unit may also include one or more processors (e.g., one or more microprocessors), one or more central processing units, CPU, one or more graphics processing units, GPU, and/or one or more signal processors (e.g., one or more digital signal processors, DSP). The computing unit may also include a physical or a virtual cluster of computers or other of the units.

In various embodiments, the computing unit includes one or more hardware and/or software interfaces and/or one or more memory units.

A memory unit may be implemented as a volatile data memory (e.g., a dynamic random access memory, DRAM, or a static random access memory, SRAM) or as a non-volatile data memory (e.g., a read-only memory, ROM, a programmable read-only memory, PROM, an erasable programmable read-only memory, EPROM, an electrically erasable programmable read-only memory, EEPROM, a flash memory or flash EEPROM, a ferroelectric random access memory, FRAM, a magnetoresistive random access memory, MRAM, or a phase-change random access memory, PCRAM).

According to a further aspect of the present embodiments, a further data processing apparatus including at least one further computing unit that is configured to carry out a computer implemented training method according to the present embodiments is provided.

According to a further aspect of the present embodiments, an X-ray imaging system is provided. The X-ray imaging system includes a C-arm, an X-ray source mounted on the C-arm, and an X-ray detector mounted on the C-arm. The X-ray imaging system includes at least one computing unit that is configured to carry out a computer implemented method for automatically determining an operation mode for the X-ray imaging system according to the present embodiments. In other words, the X-ray imaging system includes a data processing apparatus according to the present embodiments.

According to a further aspect of the present embodiments, a first computer program including first instructions is provided. When the first instructions are executed by a data processing apparatus, the first instructions cause the data processing apparatus to carry out a computer implemented method for automatically determining an operation mode for the X-ray imaging system according to the present embodiments.

According to a further aspect of the present embodiments, a second computer program including second instructions is provided. When the second instructions are executed by a data processing apparatus, the second instructions cause the data processing apparatus to carry out a computer implemented training method according to the present embodiments.

According to a further aspect of the present embodiments, a third computer program including third instructions is provided. When the third instructions are executed by an X-ray imaging system according to the present embodiments (e.g., by the at least one computing unit of the X-ray imaging system), the third instructions cause the X-ray imaging system to carry out a method for X-ray imaging according to the present embodiments.

According to a further aspect of the present embodiments, a computer-readable storage medium storing a first computer program and/or a second computer program and/or a third computer program according to the present embodiments is provided.

The first, second, and third computer program and the computer-readable storage medium are respective computer program products including the first, second, and/or third instructions.

The first, second, and/or third instructions may be provided as program code, for example. The program code may, for example, be provided as binary code or assembler and/or as source code of a programming language (e.g., C) and/or as program script (e.g., Python).

Above and in the following, the solution according to the present embodiments is described with respect to the claimed systems as well as with respect to the claimed methods. Features, advantages, or alternative embodiments herein may be assigned to the other claimed objects and vice versa. In other words, claims and embodiments for the systems may be improved with features described or claimed in the context of the respective methods. In this case, the functional features of the method are implemented by physical units of the system.

Further, above and in the following, the solution according to the present embodiments is described with respect to methods and systems for automatically determining an operation mode for an X-ray imaging system and for X-ray imaging, as well as with respect to methods and systems for training an MLM. Features, advantages, or alternative embodiments herein may be assigned to the other claimed objects and vice versa. In other words, claims and embodiments for training the MLM may be improved with features described or claimed in the context of automatically determining an operation mode for an X-ray imaging system and X-ray imaging. For example, datasets used in the methods and systems may have the same properties and features as the corresponding datasets used in the methods and systems for providing a trained MLM, and the trained MLMs provided by the respective methods and systems may be used in the methods and systems for automatically determining an operation mode for an X-ray imaging system and for X-ray imaging.

Further features and feature combinations of the present embodiments are obtained from the figures and their description as well as the claims. For example, further implementations of the present embodiments may not necessarily contain all features of one of the claims. Further implementations of the present embodiments may include features or combinations of features that are not recited in the claims.

In the following, the present embodiments will be explained in detail with reference to specific example implementations and respective schematic drawings. In the drawings, same or functionally same elements may be denoted by the same reference signs. The description of same or functionally same elements is not necessarily repeated with respect to different figures.

### BRIEF DESCRIPTION OF THE DRAWINGS

FIG. 1 shows schematically an example implementation of an X-ray imaging system according to an embodiment;

FIG. 2 shows a schematic flow diagram of an example implementation of a method for X-ray imaging according to an embodiment;

FIG. 3 shows a schematic flow diagram of an example implementation of a computer implemented training method according to an embodiment;

FIG. 4 shows a schematic block diagram of a first feature extraction module of a machine learning model (MLM) for use in an example implementation of a computer implemented method according to an embodiment during training of the first feature extraction module;

FIG. 5 shows a schematic block diagram of a second feature extraction module of an MLM for use in a further example implementation of a computer implemented method according to an embodiment during training of the second feature extraction module;

FIG. 6 shows a schematic block diagram of a trained MLM for use in a further example implementation of a computer implemented method according to an embodiment;

FIG. 7 shows a diagram depicting angulation states of a C-arm for different X-ray imaging use cases;

FIG. 8 shows schematically a multi-layer perceptron; and

FIG. 9 shows schematically a convolutional neural network.

### DETAILED DESCRIPTION

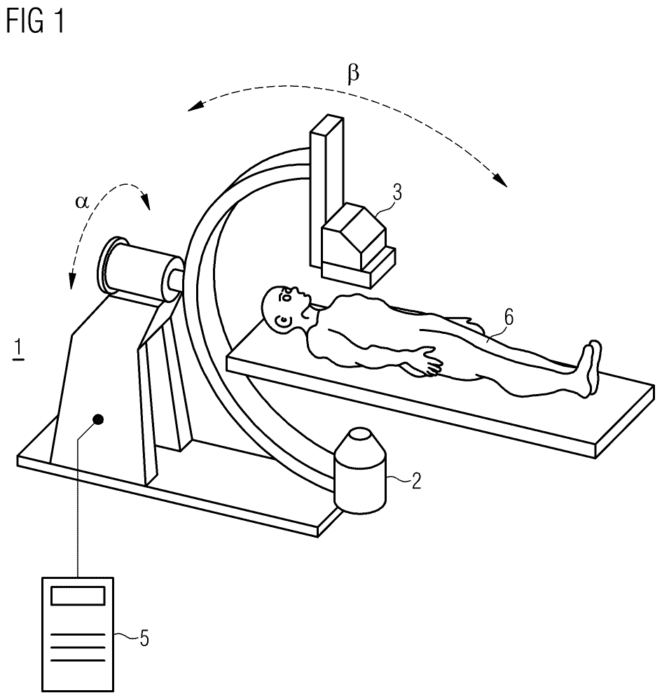

FIG. 1 shows schematically an example implementation of an X-ray imaging system 1 according to the present embodiments. The X-ray imaging system 1 includes a C-arm 4 with an X-ray source 2 and an X-ray detector 3 mounted on the C-arm 4. The C-arm 4 is configured such that the X-ray source 2 and the X-ray detector 3 may be rotated about at least two rotation axes corresponding to an angular rotation angle α and an orbital rotation angle β, respectively. This allows to adjust the C-arm 4 to different angulation states given by (α,β).

The X-ray imaging system 1 includes at least one computing unit 5 that is configured to carry out a computer implemented method for automatically determining an operation mode for the X-ray imaging system 1 according to the present embodiments.

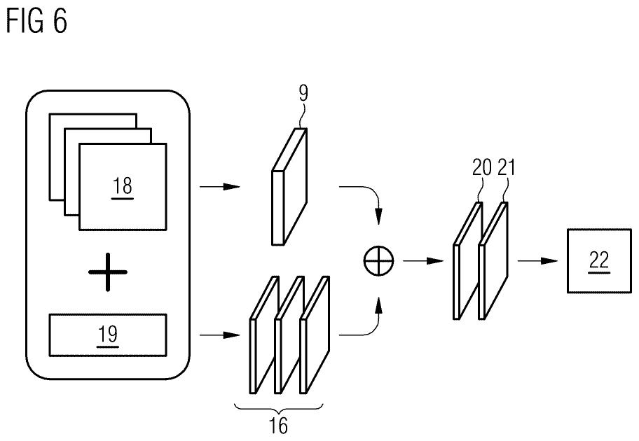

According to the computer implemented method, angulation data 19 (see FIG. 6) defining the angulation state of the C-arm 4 is received, and at least one X-ray image 18 (see FIG. 6) depicting an object 6 according to the angulation state is received. The operation mode 22 (see FIG. 6) for the X-ray imaging system 1 is selected as one of two or more predefined operation modes by applying a trained MLM for classification to input data. The input data includes the at least one X-ray image 18 and the angulation data 19.

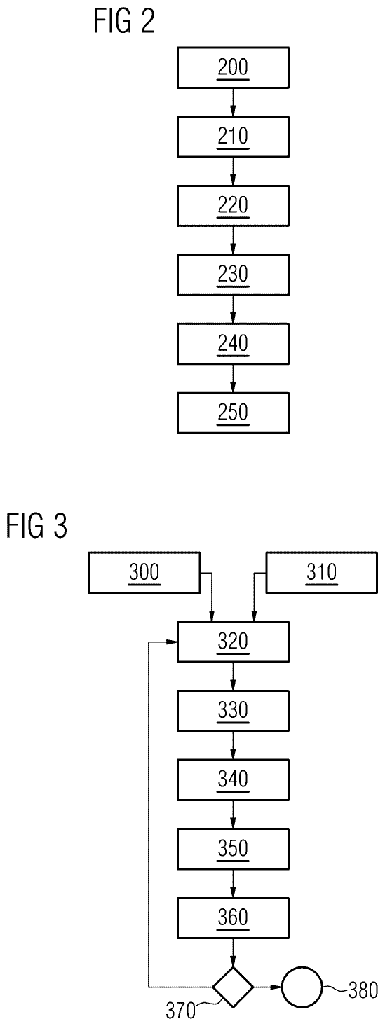

For example, a method for X-ray imaging according to the present embodiments may be carried out by the X-ray imaging system 1. A schematic flow diagram of such a method is shown in FIG. 2.

In act 200, the angulation data 19 is received, and in act 210, the X-ray imaging system 1 is configured according to at least one preliminary setting. In act 220, the at least one X-ray image 18 is received, and in act 230, the trained MLM is applied to the input data to select the operation mode 22. In act 240, the X-ray imaging system 1 is configured according to the selected operation mode 22. After that, a further X-ray image is generated according to the selected operation mode 22 by using the X-ray source 2 and the X-ray detector 3 in act 250.

FIG. 6 shows a schematic block diagram of the trained MLM for an exemplary implementation of a computer implemented method according to the present embodiments. The MLM is configured as an ANN in this implementation. The MLM includes a first feature extraction module 9 (e.g., a CNN, such as a ResNet-50) that is applied to the at least one X-ray image 18 to generate image features. The MLM includes a second feature extraction module 16 (e.g., an MLP) that is applied to the angulation data 19 to generate angulation features. The image features and the angulation features are fused (e.g., concatenated). The operation mode 22 is selected by applying at least one fully connected neural network layer 20, 21 of the MLM to the fused features.

FIG. 3 shows a schematic flow diagram of an example implementation of a computer implemented training method for training the MLM according to the present embodiments.

In act 300, the pre-trained first feature extraction module 9 is received, and in act 310, the pre-trained second feature extraction module 16 is received. The parameters of both feature extraction modules 9, 16 are frozen for training the MLM further (e.g., for training the at least one fully connected neural network layer 20, 21).

In act 320, angulation training data defining a training angulation state of the C-arm 4 is received, and in act 330, at least one training image depicting an object 6 according to the angulation state is received. In act 340, a ground truth annotation for the angulation training data and the at least one training image are received.

In act 350, a predicted operation mode for the X-ray imaging system 1 is selected as one of two or more predefined operation modes by applying the MLM to input training data that includes the at least one X-ray training image and the training angulation data. For example, training image features are generated by applying the pre-trained first feature extraction module 9 to the at least one X-ray training image, and training angulation features are generated by applying the pre-trained second feature extraction module 16 to the angulation training data. Training fused features are generated by fusing the training image features with the training angulation features. The predicted operation mode is selected by applying the at least one fully connected neural network layer 20, 21 to the fused features.

In act 360, the MLM is updated depending on the predicted operation mode and the ground truth annotation. For example, network parameters of the at least one fully connected neural network layer 20, 21 are updated depending on the predicted operation mode and the ground truth annotation.

In act 370, it is checked whether a predefined termination or convergence criterion is fulfilled. If this is the case, the trained MLM is provided in act 380. Otherwise, another training run is carried out including acts 320 to 330, for example, with a new set of X-ray training images, angulation training data, and a corresponding ground truth annotation.

Using the present embodiments, the mere utilization of image datasets is extended by integrating the angulation data of the C-arm 4. This integration is significant since the angulation state of the C-arm 4 is a key factor in medical diagnostic processes. Clinicians routinely adjust this angulation state, for example, to acquire diverse perspectives of the coronary artery, achieving differential magnification levels in radiographic imaging, and so forth.

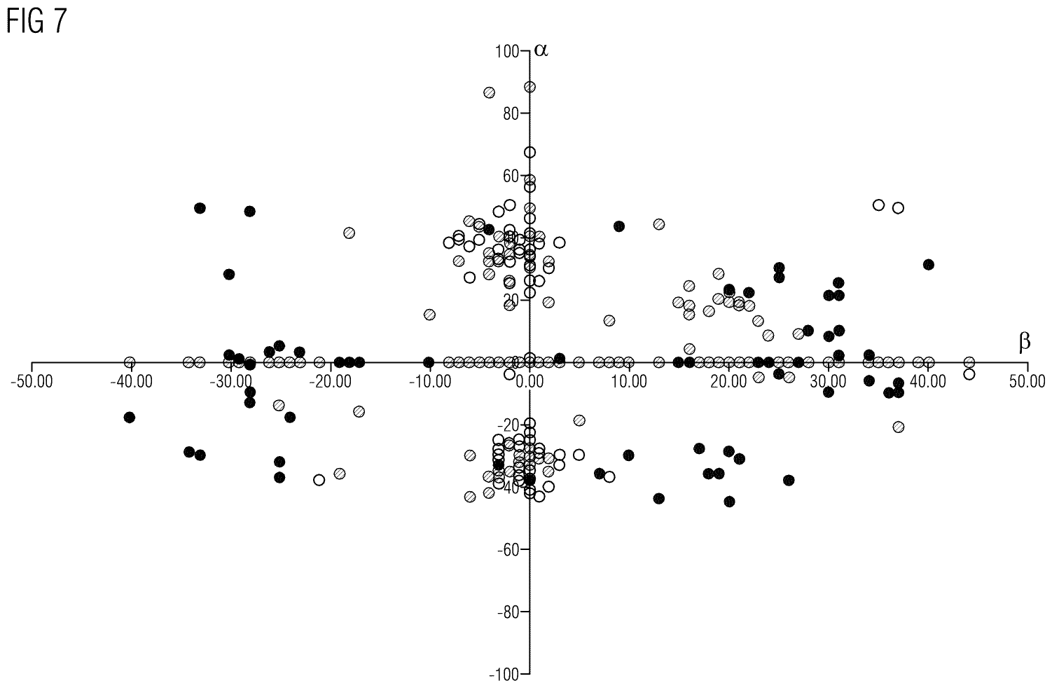

FIG. 7 shows a diagram that depicts the distribution of the angular rotation angle α and the orbital rotation angle β for multiple use cases. The black filled circles correspond to left coronary artery, LCA, imaging, the shaded circles correspond to right coronary artery, RCA, imaging, and the empty circles correspond to left ventricle, LV, imaging. As shown from the diagram, it is not promising to try to classify the different imaging use cases based on the angulation alone. The present embodiments overcome this problem by combining the X-ray images with the angulation data as input for the classification.

The angulations may be systematically categorized as left anterior oblique, LAO, and right anterior oblique, RAO, for lateral views according to the angular rotation angle α, and caudal and cranial for vertical orientations according to the orbital rotation angle β. FIG. 3b provides a graphical representation of these orientations. The angulation data may be directly obtained from the C-arm 4 controller without requiring further pre-processing. This approach enhances the classification performance significantly by incorporating a broader spectrum of diagnostic information.

In a number of implementations, the at least one training image is pre-processed X-ray images. The pre-processing may include a log transform, for example. Therein, an image is read, and a natural logarithm is applied to the image values. The logarithm compresses the displayed brightness at the bright end and expands the dark end. Ultimately, all contrasts are harmonized in the whole image without depending any longer on the background. Alternatively or in addition, the pre-processing may include a square root transform. Therein, the image is read, and the square root of the image values is computed. The focus of this is to harmonize the noise in the whole image, which originally follows the counting statistics in the raw image. This helps when performing a filtering task, for example. Alternatively or in addition, the pre-processing may include a scatter correction. Therein, the image is read, and a scatter offset value is subtracted from the image. To obtain this offset, one may first consider a portion of the collimator in the image. The mean value of this collimator region is calculated and represents the offset. If the log transform is also used, the logarithm is, for example, applied to the scatter corrected image.

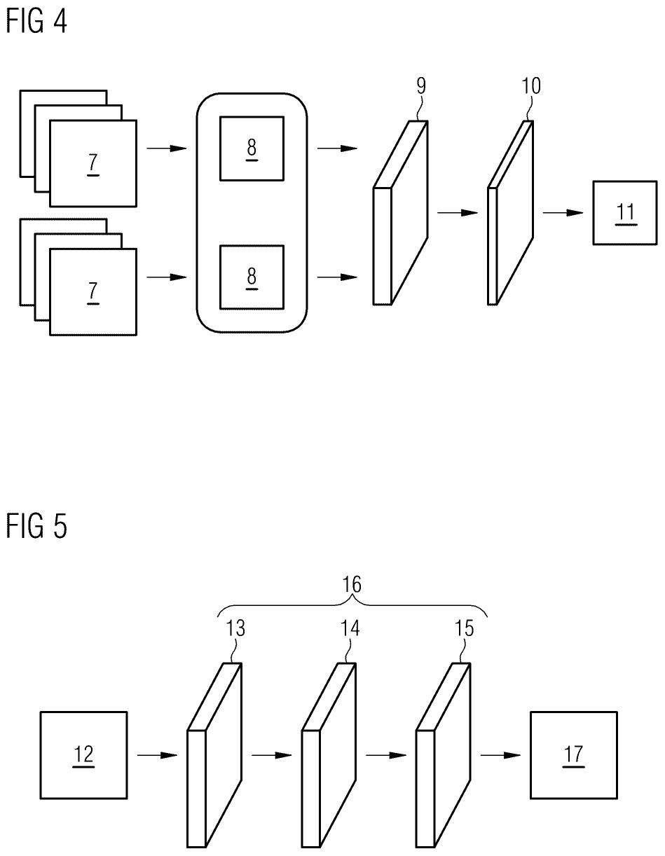

In a number of implementations, a pre-trained Resnet-50 architecture is employed for the first feature extraction module 9. This Resnet-50 model is, for example, pre-trained on the ImageNet dataset. Further training images 7 or pre-processed further training images 8 are provided as input to the Resnet-50 for feature extraction, and the extracted features are then passed through a fully connected layer 10 for classification, as depicted schematically in FIG. 4.

For example, a batch size of 2 and a learning rate of 104 may be used as hyperparameters for training the first feature extraction module 9. For multi-class classification, a categorical cross entropy with a Softmax activation function may be used as the loss function, which may be optimized by using an Adam optimizer. Early stopping may be used with a patience value of 25 while monitoring the validation loss to avoid overfitting.

In a number of implementations, an MLP, for example, consisting of three layers 13, 14, 15 is used for the second feature extraction module 16. The first two layers 13, 14 are used for feature extraction from further angulation training data 12, and the final layer 15 is used for classification, as depicted schematically in FIG. 5.

For the training of the second feature extraction module 16, one may use a batch size of 100 and a learning rate of 0.01. The categorical-cross entropy with a Softmax activation may be used as a loss function, and the Adam optimizer may also be used here. Also early stopping with a patience value of 25, while monitoring the validation loss, may be used.

The full MLM may thus leverage the principles of transfer learning. The pre-trained feature extraction modules 9, 16 are integrated, as shown in FIG. 6. Then, the at least one fully connected neural network layer 20, 21 is trained as described above. The fully connected layer 10 is then not required anymore. Specifically, when training the at least one fully connected neural network layer 20, 21, the Resnet-50 layers are frozen, maintaining the pre-existing weights acquired from the image data model, rendering these layers non-trainable. In a parallel manner, the MLP layers are similarly frozen and loaded with pre-trained weights from the angulation data model. The resultant feature vectors from both models are concatenated and then fed into the at least one fully connected neural network layer 20, 21. This process culminates in a final fully connected layer 21 that is dedicated to the task of classification. For example, the same hyperparameters and loss function as used for training the first feature extraction module 9 may be used.

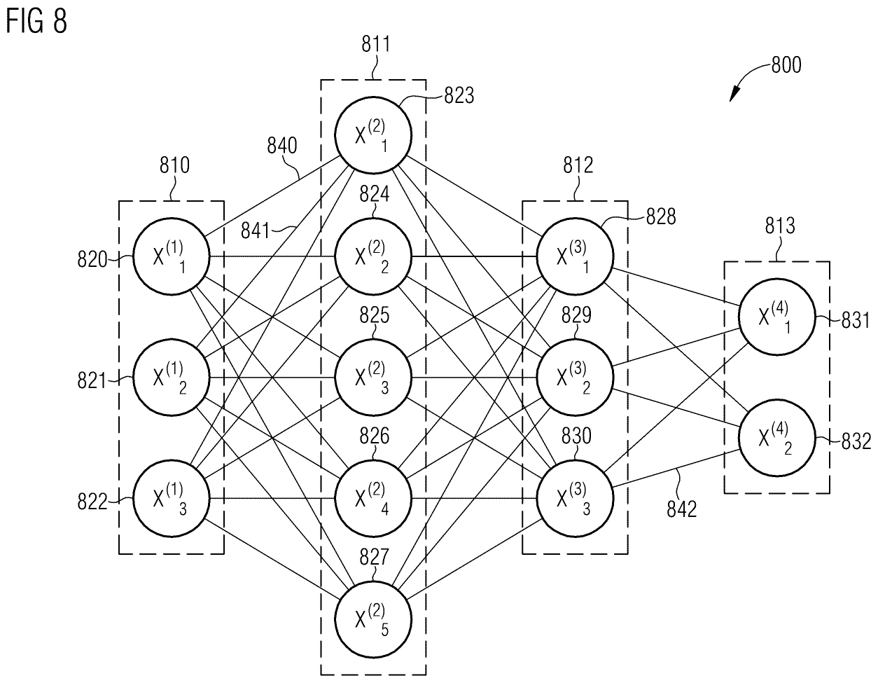

As mentioned above, the second feature extraction module 16 may be an MLP. FIG. 8 displays an embodiment of an ANN 800 that is, for example, configured as an MLP. The ANN 800 includes nodes 820, . . . , 832 and edges 840, . . . , 842, where each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832. It is, however, also possible that the first node 820, . . . , 832 and the second node 820, . . . , 832 are identical. For example, in FIG. 8, the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as ingoing edge for the second node 820, . . . , 832 and as outgoing edge for the first node 820, . . . , 832.

In this example, the nodes 820, . . . , 832 of the artificial neural network 800 may be arranged in layers 810, . . . , 813, where the layers may include an intrinsic order introduced by the edges 840, . . . , 842 between the nodes 820, . . . , 832. For example, edges 840, . . . , 842 may exist only between neighboring layers of nodes. In the displayed example, there is an input layer 810 including only nodes 820, . . . , 822 without an incoming edge, an output layer 813 including only nodes 831, 832 without outgoing edges, and hidden layers 811, 812 in between the input layer 810 and the output layer 813. In general, the number of hidden layers 811, 812 may be chosen arbitrarily. In an MLP, this number is at least one. The number of nodes 820, . . . , 822 within the input layer 810 may relate to the number of input values of the artificial neural network 800, and the number of nodes 831, 832 within the output layer 813 may relate to the number of output values of the artificial neural network 800.

For example, a real number may be assigned as a value to every node 820, . . . , 832 of the artificial neural network 800. For example, x⁽ⁿ⁾ᵢ denotes the value of the i-th node 820, . . . , 832 of the n-th layer 810, . . . , 813. The values of the nodes 820, . . . , 822 of the input layer 810 are equivalent to the input values of the artificial neural network 800. The values of the nodes 831, 832 of the output layer 813 are equivalent to the output value of the artificial neural network 800. Further, each edge 840, . . . , 842 may include a weight being a real number. For example, the weight is a real number within the interval [−1, 1] or within the interval [0, 1]. For example, w^{(m,n)}_{i,j} denotes the weight of the edge between the i-th node 820, . . . , 832 of the m-th layer 810, . . . , 813 and the j-th node 820, . . . , 832 of the n-th layer 810, . . . , 813. Further, the abbreviation w⁽ⁿ⁾_{i,j} is defined for the weight w^{(n,n+1)}_{i,j}.

For example, to calculate the output values of the neural network 800, the input values are propagated through the neural network 800. For example, the values of the nodes 820, . . . , 832 of the (n+1)-th layer 810, . . . , 813 may be calculated based on the values of the nodes 820, . . . , 832 of the n-th layer 810, . . . , 813 by

$${x}_{j}^{( n + 1 )} = f ( {\sum }_{i} {x}_{i}^{( n )} {w}_{i , j}^{( n )} ) .$$

Herein, the function f is denoted as transfer function or activation function. Known transfer functions are step functions, the sigmoid functions (e.g., the logistic function), the generalized logistic function, the hyperbolic tangent, the arctangent function, the error function, the smoothstep function, or rectifier functions. The transfer function is mainly used for normalization purposes.

For example, the values are propagated layer-wise through the neural network 800. Values of the input layer 810 are given by the input of the neural network 800; values of the first hidden layer 811 may be calculated based on the values of the input layer 810 of the neural network 800; values of the second hidden layer 812 may be calculated based in the values of the first hidden layer 811; and so forth.

In order to set the values w^{(m,n)}_{i,j} for the edges, the neural network 800 is to be trained using training data. For example, training data includes training input data and training output data (e.g., denoted as ti). For a training step, the neural network 800 is applied to the training input data to generate calculated output data. For example, the training data and the calculated output data include a number of values. The number is equal to the number of nodes of the output layer. For example, a comparison between the calculated output data and the training data is used to recursively adapt the weights within the neural network 800 (e.g., backpropagation algorithm). For example, the weights are changed according to

$${{w}^{′}}_{i , j}^{( n )} = {w}_{i , j}^{( n )} - {γδ}_{j}^{( n )} {x}_{i}^{( n )} ,$$

where γ is a predefined learning rate, and the numbers δ⁽ⁿ⁾ⱼ may be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

based on δ⁽ⁿ⁺¹⁾ⱼ, if the (n+1)-th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)-th layer is the output layer 813. f′ is the first derivative of the activation function, and t⁽ⁿ⁺¹⁾ⱼ is the comparison training value for the j-th node of the output layer 813.

As mentioned above, the first feature extraction module 9 may be a CNN. A CNN is an ANN that uses a convolution operation instead of general matrix multiplication in at least one of its layers. These layers are denoted as convolutional layers. For example, a convolutional layer performs a dot product of one or more convolution kernels with the convolutional layer's input data, where the entries of the one or more convolution kernel are parameters or weights that may be adapted by training. For example, one may use the Frobenius inner product and the ReLU activation function. A convolutional neural network may include additional layers (e.g., pooling layers, fully connected layers, and/or normalization layers).

By using convolutional neural networks, the input may be processed in a very efficient way because a convolution operation based on different kernels may extract various image features so that by adapting the weights of the convolution kernel, the relevant image features may be found during training. Further, based on the weight-sharing in the convolutional kernels, fewer parameters are to be trained, which prevents overfitting in the training phase and allows to have faster training or more layers in the network, improving the performance of the network.

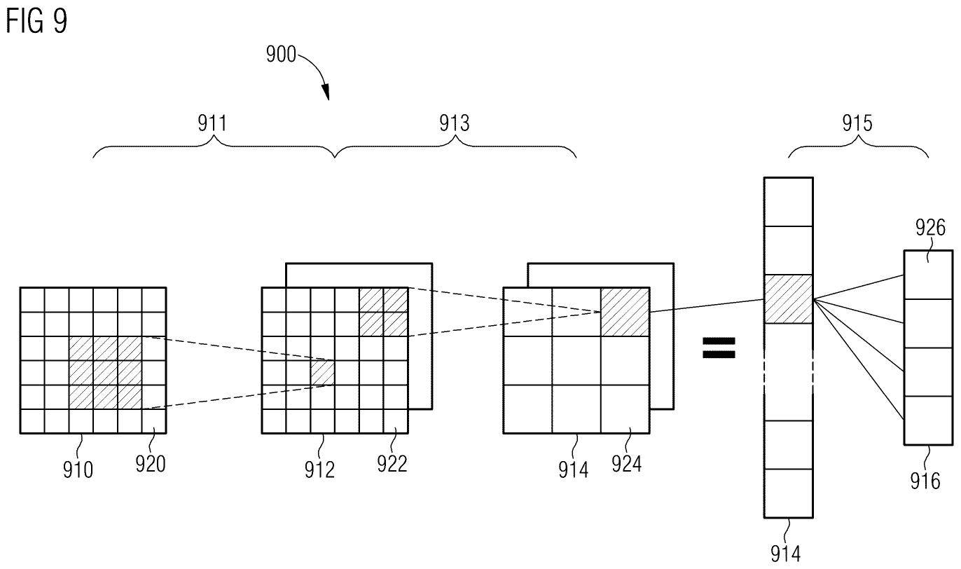

FIG. 9 displays an example embodiment of a convolutional neural network 900. In the displayed embodiment, the convolutional neural network 900 includes an input node layer 910, a convolutional layer 911, a pooling layer 913, a fully connected layer 914, and an output node layer 916, as well as hidden node layers 912, 914. Alternatively, the convolutional neural network 900 may include a number of convolutional layers 911, a number of pooling layers 913, and/or a number of fully connected layers 915, as well as other types of layers. The order of the layers may be chosen arbitrarily. Fully connected layers 915 may be used as the last layers before the output layer 916.

For example, within a convolutional neural network 900, nodes 920, 922, 924 of a node layer 910, 912, 914 may be considered to be arranged as a d-dimensional matrix or as a d-dimensional image. For example, in the two-dimensional case, the value of the node 920, 922, 924 indexed with i and j in the n-th node layer 910, 912, 914 may be denoted as x(n)[i, j]. However, the arrangement of the nodes 920, 922, 924 of one node layer 910, 912, 914 does not have an effect on the calculations executed within the convolutional neural network 900 as such, since these are given solely by the structure and the weights of the edges.

A convolutional layer 911 is a connection layer between an anterior node layer 910 with node values x(n−1) and a posterior node layer 912 with node values x(n). For example, a convolutional layer 911 is characterized by the structure and the weights of the incoming edges forming a convolution operation based on a certain number of kernels. For example, the structure and the weights of the edges of the convolutional layer 911 are chosen such that the values x(n) of the nodes 922 of the posterior node layer 912 are calculated as a convolution x(n)=K*x(n−1) based on the values x(n−1) of the nodes 920 anterior node layer 910, where the convolution * is defined in the two-dimensional case as

$${x}^{( n )} [ i , j ] = ( K * {x}^{( n - 1 )} ) [ i , j ] = {\sum }_{i ′} {\sum }_{j ′} K [ {i}^{′} , {j}^{′} ] \cdot {x}^{( n - 1 )} [ i - {i}^{′} , j - {j}^{′} ] .$$

Herein, the kernel K is a d-dimensional matrix (e.g., in the present example a two-dimensional matrix) that may be small compared to the number of nodes 920, 922 (e.g., a 3×3 matrix or a 5×5 matrix). For example, this implies that the weights of the edges in the convolution layer 911 are not independent, but chosen such that the weights produce the convolution equation. For example, for a kernel being a 3×3 matrix, there are only 9 independent weights. Each entry of the kernel matrix corresponds to one independent weight, irrespectively of the number of nodes 920, 922 in the anterior node layer 910 and the posterior node layer 912.

In general, convolutional neural networks 900 use node layers 910, 912, 914 with a plurality of channels, for example, due to the use of a plurality of kernels in convolutional layers 911. In those cases, the node layers may be considered as (d+1)-dimensional matrices. The first dimension indexes the channels. The action of a convolutional layer 911 is then in a two-dimensional example defined as

$${x}_{b}^{( n )} [ i , j ] = {\sum }_{a} ( {K}_{a , b} * {x}_{a}^{( n - 1 )} [ i , j ] = {\sum }_{a} {\sum }_{i ′} {\sum }_{j ′} {K}_{a , b} [ {i}^{′} , {j}^{′} ] \cdot {x}_{a}^{( n - 1 )} [ i - {i}^{′} , j - {j}^{′} ] ,$$

where xₐ⁽ⁿ⁾ corresponds to the a-th channel of the anterior node layer 910, x_{b}⁽ⁿ⁾ corresponds to the b-th channel of the posterior node layer 912, and K_{a,b} corresponds to one of the kernels. If a convolutional layer 911 acts on an anterior node layer 910 with A channels and outputs a posterior node layer 912 with B channels, there are A·B independent d-dimensional kernels K_{a,b}.

In general, in convolutional neural networks 900 activation functions may be used. In this embodiment, ReLU (e.g., rectified linear unit) is used, with R (z)=max (0, z), so that the action of the convolutional layer 911 in the two-dimensional example is

$${x}_{b}^{( n )} [ i , j ] = R ( {\sum }_{a} ( {K}_{a , b} * {x}_{a}^{( n - 1 )} [ i , j ] ) = R ( {\sum }_{a} {\sum }_{{i}^{′}} {\sum }_{{j}^{′}} {K}_{a , b} [ {i}^{′} , {j}^{′} ] \cdot {x}_{a}^{( n - 1 )} [ i - {i}^{′} , j - {j}^{′} ] ) .$$

It is also possible to use other activation functions (e.g., exponential linear unit (ELU), LeakyReLU, Sigmoid, Tanh, or Softmax).

In the displayed embodiment, the input layer 910 includes 36 nodes 920, arranged as a two-dimensional 6×6 matrix. The first hidden node layer 912 includes 72 nodes 922, arranged as two two-dimensional 6×6 matrices. Each of the two matrices is the result of a convolution of the values of the input layer with a 3×3 kernel within the convolutional layer 911. Equivalently, the nodes 922 of the first hidden node layer 912 may be interpreted as arranged as a three-dimensional 2×6×6 matrix, where the first dimension corresponds to the channel dimension.

An advantage of using convolutional layers 911 is that spatially local correlation of the input data may be exploited by enforcing a local connectivity pattern between nodes of adjacent layers (e.g., by each node being connected to only a small region of the nodes of the preceding layer).

A pooling layer 913 is a connection layer between an anterior node layer 912 with node values x(n−1) and a posterior node layer 914 with node values x(n). For example, a pooling layer 913 may be characterized by the structure and the weights of the edges and the activation function forming a pooling operation based on a non-linear pooling function f. For example, in the two-dimensional case, the values x(n) of the nodes 924 of the posterior node layer 914 may be calculated based on the values x(n−1) of the nodes 922 of the anterior node layer 912 as

$${x}_{b}^{( n )} [ i , j ] = f ( {x}_{b}^{( n - 1 )} [ {id}_{1} , {jd}_{2} ] , … , {x}_{b}^{( n - 1 )} [ ( i + 1 ) {d}_{1} - 1 , ( j + 1 ) {d}_{2} - 1 ] ) .$$

In other words, by using a pooling layer 913, the number of nodes 922, 924 may be reduced by replacing a number d1·d2 of neighboring nodes 922 in the anterior node layer 912 with a single node 922 in the posterior node layer 914 being calculated as a function of the values of the number of neighboring nodes. For example, the pooling function f may be the max-function, the average, or the L2-Norm. For example, for a pooling layer 913, the weights of the incoming edges are fixed and are not modified by training.

The advantage of using a pooling layer 913 is that the number of nodes 922, 924 and the number of parameters is reduced. This leads to the amount of computation in the network being reduced and to a control of overfitting.

In the displayed embodiment, the pooling layer 913 is a max-pooling layer, replacing four neighboring nodes with only one node. The value is the maximum of the values of the four neighboring nodes. The max-pooling is applied to each d-dimensional matrix of the previous layer. In this embodiment, the max-pooling is applied to each of the two two-dimensional matrices, reducing the number of nodes from 72 to 18.

In general, the last layers of a convolutional neural network 900 may be fully connected layers 915. A fully connected layer 915 is a connection layer between an anterior node layer 914 and a posterior node layer 916. A fully connected layer 913 may be characterized by the fact that a majority of (e.g., all) edges between nodes 914 of the anterior node layer 914 and the nodes 916 of the posterior node layer are present. The weight of each of these edges may be adjusted individually.

In this embodiment, the nodes 924 of the anterior node layer 914 of the fully connected layer 915 are displayed both as two-dimensional matrices, and additionally as non-related nodes, indicated as a line of nodes. The number of nodes was reduced for a better presentability. This operation is also denoted as flattening. In this embodiment, the number of nodes 926 in the posterior node layer 916 of the fully connected layer 915 is smaller than the number of nodes 924 in the anterior node layer 914. Alternatively, the number of nodes 926 may be equal or larger.

Further, in this embodiment, the Softmax activation function is used within the fully connected layer 915. By applying the Softmax function, the sum the values of all nodes 926 of the output layer 916 is 1, and all values of all nodes 926 of the output layer 916 are real numbers between 0 and 1. For example, if using the convolutional neural network 900 for categorizing input data, the values of the output layer 916 may be interpreted as the probability of the input data falling into one of the different categories.

For example, convolutional neural networks 900 may be trained based on the backpropagation algorithm. For preventing overfitting, methods of regularization may be used (e.g., dropout of nodes 920, . . . , 924, stochastic pooling, use of artificial data, weight decay based on the L1 or the L2 norm, or max norm constraints).

In known approaches, users select imaging parameters via a manual, stepwise process. After identifying the broad target area, such as the heart, the imaging system may offer a suite of organ-specific programs. The user then fine-tunes this selection, pinpointing the exact coronary segment or branch for intervention, such as the left anterior descending artery or the right coronary artery, and aligning the program accordingly. This iterative process introduces avenues for oversight and may prolong the procedure, potentially impacting patient outcomes, and using generalized programs leads to less optimal image quality and unnecessary radiation exposure. The present embodiments overcome the drawbacks.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for automatically determining an operation mode for an X-ray imaging system that comprises an X-ray source and an X-ray detector mounted on a C-arm, the computer-implemented method comprising:
receiving angulation data defining an angulation state of the C-arm;
receiving at least one X-ray image depicting an object according to the angulation state; and
selecting the operation mode for the X-ray imaging system as one of two or more predefined operation modes, the selecting comprising applying a trained machine learning model (MLM) for classification to input data, the input data comprising the at least one X-ray image and the angulation data.

2. The computer-implemented method of claim 1, further comprising:
generating an information message informing a user about the selected operation mode;
automatically configurating the X-ray imaging system according to the selected operation mode; or
a combination thereof.

3. The computer-implemented method of claim 2, wherein automatically configuring the X-ray imaging system according to the selected operation mode comprises setting a value of an X-ray exposure time assigned to the selected operation mode, setting a value of a frame rate assigned to the selected operation mode, setting at least one image processing parameter assigned to the selected operation mode, activating or deactivating a function of the X-ray imaging system assigned to the selected operation mode, or any combination thereof.

4. The computer-implemented method of claim 1, further comprising:
generating image features, the generating of the image features comprising applying a first feature extraction module of the MLM to the at least one X-ray image; and
generating angulation features, the generating of the angulation features comprising applying a second feature extraction module of the MLM to the angulation data;
wherein the operation mode for the X-ray imaging system is selected depending on the image features and the angulation features.

5. The computer-implemented method of claim 4, further comprising:
generating fused features, the generating of the fused features comprising fusing the image features with the angulation features,
wherein the operation mode of the X-ray imaging system is selected depending on the fused features.

6. The computer-implemented method of claim 5, wherein selecting the operation mode of the X-ray system comprises applying at least one fully connected neural network layer of the MLM to the fused features.

7. The computer-implemented method of claim 4, wherein the first feature extraction module comprises a convolutional neural network, a residual neural network, or the convolutional neural network and the residual neural network.

8. The computer-implemented method of claim 4, wherein the second feature extraction module comprises a multi-layer perceptron.

9. The computer-implemented method of claim 1, wherein the angulation data comprises an angular rotation angle and an orbital rotation angle of the C-arm.

10. The computer-implemented method of claim 1, wherein the two or more predefined operation modes comprise a first operation mode for imaging right coronary arteries, a second operation mode for imaging left coronary arteries, a third operation mode for imaging a left ventricle, or any combination thereof.

11. A method for X-ray imaging using an X-ray imaging system that comprises an X-ray source and an X-ray detector mounted on a C-arm, the method comprising:
determining angulation data defining an angulation state of the C-arm;
configuring the X-ray imaging system according to at least one preliminary setting;
generating at least one X-ray image depicting an object according to the angulation state and according to the at least one preliminary setting using the X-ray source and the X-ray detector;
automatically determining an operation mode for the X-ray imaging system, the automatically determining comprising selecting the operation mode for the X-ray imaging system as one of two or more predefined operation modes, the selecting comprising applying a trained machine learning model (MLM) for classification to input data, the input data comprising the at least one X-ray image and the angulation data;
configuring the X-ray imaging system according to the selected operation mode for the X-ray imaging system; and
generating a further X-ray image according to the selected operation mode for the X-ray imaging system using the X-ray source and the X-ray detector.

12. A computer-implemented training method for training a machine learning model (MLM) for classification for use in a computer-implemented method for automatically determining an operation mode for an X-ray imaging system that comprises an X-ray source and an X-ray detector mounted on a C-arm, the computer-implemented training method comprising:
receiving angulation training data defining a training angulation state of the C-arm;
receiving at least one training image depicting an object according to the training angulation state;
receiving a ground truth annotation for the angulation training data and the at least one training image;
selecting a predicted operation mode for the X-ray imaging system as one of two or more predefined operation modes, the selecting comprising applying the MLM to input training data that comprises the at least one X-ray training image and the training angulation data; and
updating the MLM depending on the selected predicted operation mode and the ground truth annotation.

13. The computer-implemented training method of claim 12, further comprising:
generating training image features, the generating of the training image features comprising applying a pre-trained first feature extraction module of the MLM to the at least one X-ray training image;
generating training angulation features, the generating of the training angulation features comprising applying a pre-trained second feature extraction module of the MLM to the angulation training data;
generating training fused features, the generating of the training fused features comprising fusing the training image features with the training angulation features, wherein selecting the predicted operation mode for the X-ray imaging system comprises applying at least one fully connected neural network layer of the MLM to the training fused features; and
updating network parameters of the at least one fully connected neural network layer depending on the predicted operation mode and the ground truth annotation.

14. A data processing apparatus comprising:
at least one computing unit configured to automatically determine an operation mode for an X-ray imaging system that comprises an X-ray source and an X-ray detector mounted on a C-arm, the at least one computing unit being configured to automatically determine the operation mode of the X-ray imaging system comprising the at least one computing unit being configured to:
receive angulation data defining an angulation state of the C-arm;
receive at least one X-ray image depicting an object according to the angulation state; and
select the operation mode for the X-ray imaging system as one of two or more predefined operation modes, the selection comprising application of a trained machine learning model (MLM) for classification to input data, the input data comprising the at least one X-ray image and the angulation data;
at least one further computing unit configured to train the MLM, the at least one further computing unit being configured to train the MLM comprising the at least one further computing unit being configured to:
receive angulation training data defining a training angulation state of the C-arm;
receive at least one training image depicting an object according to the training angulation state;
receive a ground truth annotation for the angulation training data and the at least one training image;
select a predicted operation mode for the X-ray imaging system as one of two or more predefined operation modes, the selecting comprising applying the MLM to input training data that comprises the at least one X-ray training image and the training angulation data; and
updating the MLM depending on the selected predicted operation mode and the ground truth annotation.

15. An X-ray imaging system comprising:
an X-ray source; and
an X-ray detector mounted on a C-arm; and
at least one computing unit configured to automatically determine an operation mode for an X-ray imaging system that comprises an X-ray source and an X-ray detector mounted on a C-arm, the at least one computing unit being configured to automatically determine the operation mode for the X-ray imaging system comprising the at least one computing unit being configured to:
receive angulation data defining an angulation state of the C-arm;
receive at least one X-ray image depicting an object according to the angulation state; and
select the operation mode for the X-ray imaging system as one of two or more predefined operation modes, the selection comprising application of a trained machine learning model (MLM) for classification to input data, the input data comprising the at least one X-ray image and the angulation data.
